(19) United States
(12) Patent Application Publication
(10) Pub. No.: US 2018/0334704 A1
(43) Pub. Date: Nov. 22, 2018

(54) METHODS FOR DETECTING MALIGNANT COLON CONDITIONS

(71) Applicant: Coyote Diagnostics Lab (Beijing) Co., Ltd., Beijing (CN)

(72) Inventor: Patrick Pak Tak Leong, Campbell, CA (US)

(21) Appl. No.: 15/599,013

(22) Filed: May 18, 2017

(51) Int. Cl. C12Q 1/68; G06F 19/24; G06F 19/22

(52) U.S. Cl. CPC C12Q 1/689; G06F 19/24; C12Q 2600/118; C12Q 1/6886; G06F 19/22

(57) ABSTRACT

The present disclosure provides methods and systems directed to detecting malignant colon conditions. A method for identifying or monitoring a progression or regression of a malignant colon condition in a subject comprises processing a biological sample obtained from the subject to generate data indicative of a distribution of a plurality of populations of microbes of different types in the biological sample. A presence, absence, or relative amount of individual populations of microbes of the plurality of populations of microbes may be indicative of a malignant colon condition. Next, a trained algorithm may be to process the data to determine a presence, absence, or relative amount of the individual populations of microbes. Next, based on the presence, absence, or relative amount, the subject may be identified as having the malignant colon condition, such as, for example, in a report.

### BACKGROUND

Colorectal cancer (cancer of the colon and rectum) is the second leading cause of cancer-related death in the U.S. Colorectal cancer screening is often recommended for adults 50 years old or older to screen for either malignant growths or precancerous polyps which can be removed before becoming cancerous. Common screening tests include colonoscopy, flexible sigmoidoscopy, fecal occult blood test (FOBT), and fecal immunochemical test (FIT). However, because of costs and the invasive nature of colonoscopy and sigmoidoscopy, only about 70% of adults recommended for screening will actually do so.

Widespread screening of asymptomatic adults is especially critical for colorectal cancer because colorectal cancer risk may be significantly reduced by removal of precancerous polyps (e.g., during a colonoscopy procedure) and colorectal cancer is highly curable by surgical resection and other therapeutic interventions if detected in its early stages. About 60 percent of colorectal cancer deaths may be prevented if all adults age 50 or older and other individuals with elevated risk (e.g., adults under age 50 with a family history of colorectal cancer) are regularly screened using an accurate non-invasive screening test and appropriately treated if needed. Thus, there exists a need for rapid, accurate screening methods for colorectal cancer that are non-invasive and cost-effective.

### SUMMARY

The present disclosure provides methods, systems, and kits for detecting malignant colon conditions by processing biological samples indicative of a distribution of a plurality of populations of microbes of different types. Biological samples (e.g., stool samples) obtained from subjects may be analyzed to measure microbiome distributions. Such subjects may include subjects with malignant colon conditions (e.g., colorectal cancer) and subjects without these malignant colon conditions.

In an aspect, disclosed herein is a method for identifying or monitoring a progression or regression of a malignant colon condition in a subject, the method comprising: (a) processing a biological sample obtained from said subject to generate data indicative of a distribution of a plurality of populations of microbes of different types in said biological sample, wherein a presence, absence, or relative amount of individual populations of microbes of said plurality of populations of microbes is indicative of a malignant colon condition; (b) using a trained algorithm to process said data indicative of said distribution of said plurality of populations of microbes to determine a presence, absence, or relative amount of said individual populations of microbes of said plurality of populations of microbes in said biological sample, which trained algorithm is configured to identify said malignant colon condition with an accuracy of at least 90% for at least 100 independent samples; (c) based on said presence, absence, or relative amount of said individual populations of microbes of said plurality of populations of microbes determined in (b), identifying said subject as having said malignant colon condition with an accuracy of at least about 90%; and (d) electronically outputting a report that identifies or provides an indication of said progression or regression of said malignant colon condition in said subject.

In some embodiments, said biological sample is independent of samples used to train said trained algorithm.

In some embodiments, said trained algorithm is configured to identify said malignant colon condition with a negative predictive value (NPV) of at least about 90%. In some embodiments, said NPV is at least about 95%. In some embodiments, said trained algorithm is configured to identify said malignant colon condition with a positive predictive value (PPV) of at least about 70%. In some embodiments, said PPV is at least about 80%. In some embodiments, said PPV is as at least about 90%. In some embodiments, said PPV is as at least about 95%.

In some embodiments, said trained algorithm is configured to identify said malignant colon condition with a clinical sensitivity of at least about 90%. In some embodiments, said clinical sensitivity is at least about 95%. In some embodiments, said clinical sensitivity at least about 99%.

In some embodiments, said trained algorithm is configured to identify said malignant colon condition with an Area Under Curve (AUC) of at least about 0.90. In some embodiments, said AUC is at least about 0.95. In some embodiments, said AUC is at least about 0.99.

In some embodiments, said subject does not display a benign or malignant colon condition. In some embodiments, said biological sample is feces. In some embodiments, said trained algorithm is trained with at least 200 independent training samples. In some embodiments, said trained algorithm is trained with at least 250 independent training samples. In some embodiments, said trained algorithm is trained with at least 300 independent training samples.

In some embodiments, said trained algorithm is trained with no more than 200 independent training samples associated with presence of said malignant colon condition. In some embodiments, said trained algorithm is trained with no more than 100 independent training samples associated with presence of said malignant colon condition. In some embodiments, said trained algorithm is trained with no more than 50 independent training samples associated with presence of said malignant colon condition.

In some embodiments, said trained algorithm is trained with a first number of independent training samples associated with presence of said malignant colon condition and a second number of independent training samples associated with absence of said malignant colon condition, wherein the first number is no more than the second number.

In some embodiments, (a) comprises (i) subjecting said biological sample to conditions that are sufficient to isolate said plurality of populations of microbes, and (ii) identifying said presence, absence, or relative amount of said individual populations of microbes of said plurality of populations of microbes. In some embodiments, the method further comprises extracting nucleic acid molecules from said biological sample, and subjecting said nucleic acid molecules to sequencing to identify said presence, absence, or relative amount of said individual populations of microbes of said plurality of populations of microbes. In some embodiments, said sequencing is massively parallel sequencing. In some embodiments, said sequencing comprises nucleic acid amplification. In some embodiments, said nucleic acid amplification is polymerase chain reaction (PCR). In some embodiments, said sequencing comprises use of simultaneous reverse transcription (RT) and polymerase chain reaction (PCR). In some embodiments, the method further comprises using probes configured to selectively enrich nucleic acid molecules corresponding to said individual populations of microbes. In some embodiments, said probes are nucleic acid primers. In some embodiments, said probes have sequence complementarity with nucleic acid sequences from said individual populations of microbes.

In some embodiments, said plurality of populations of microbes comprise at least 5 different populations of microbes. In some embodiments, said plurality of populations of microbes comprise at least 10 different populations of microbes. In some embodiments, said at least 5 different populations microbes are different species of microbes. In some embodiments, said at least 5 different species of microbes comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. In some embodiments, said plurality of populations of microbes comprise one or more members selected from the group consisting of Porphyromonas, Prevotella, Peptostreptococcus, Lachnospiraceae, and Parvimonas.

In some embodiments, said biological sample is processed to identify a distribution of a plurality of populations of microbes in said biological sample without any nucleic acid extraction. In some embodiments, said report is presented on a graphical user interface of an electronic device of a user. In some embodiments, said user is said subject.

In some embodiments, said malignant colon condition is colorectal cancer. In some embodiments, the method further comprises identifying a stage of said colorectal cancer.

In some embodiments, said trained algorithm comprises a supervised machine learning algorithm. In some embodiments, said supervised machine learning algorithm comprises a Random Forest, a support vector machine (SVM), a neural network, or a deep learning algorithm.

In some embodiments, the method further comprises, upon identifying said subject as having said malignant colon condition, providing said subject with a therapeutic intervention. In some embodiments, said therapeutic intervention comprises recommending said subject for a secondary clinical test to confirm a diagnosis of said malignant colon condition. In some embodiments, said secondary clinical test comprises a colonoscopy, a biopsy, a blood test, a fecal immunochemical test (FIT), a computed tomography (CT) scan, a magnetic resonance imaging (MRI) scan, an ultrasound scan, a chest X-ray, a positron emission tomography (PET) scan, or a PET-CT scan.

In some embodiments, the method further comprises treating said subject upon identifying said subject as having said malignant colon condition.

In some embodiments, the method further comprises monitoring a course of treatment for treating said malignant colon condition in said subject, wherein said monitoring comprises assessing said malignant colon condition in said subject at two or more time points, wherein said assessing is based at least on said presence, absence, or relative amount of said individual populations of microbes of said plurality of populations of microbes determined in (b) at each of said two or more time points. In some embodiments, a difference in said presence, absence, or relative amount of said individual populations of microbes of said plurality of populations of microbes determined in (b) between said two or more time points is indicative of one or more clinical indications selected from the group consisting of: (i) a diagnosis of said malignant colon condition in said subject, (ii) a prognosis of said malignant colon condition in said subject, (iii) a progression of said malignant colon condition in said subject, (iv) a regression of said malignant colon condition in said subject, (v) an efficacy of said course of treatment for treating said malignant colon condition in said subject, and (vi) a resistance of said malignant colon condition toward said course of treatment for treating said malignant colon condition in said subject.

In some embodiments, said processing comprises assaying said biological sample using probes that are selected for said plurality of populations of microbes. In some embodiments, said plurality of populations of microbes comprise at least 5 different populations of microbes. In some embodiments, said plurality of populations of microbes comprise at least 10 different populations of microbes. In some embodiments, said at least 5 different populations microbes are different species of microbes. In some embodiments, said at least 5 different species of microbes comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. In some embodiments, said plurality of populations of microbes comprise one or more members selected from the group consisting of Porphyromonas, Prevotella, Peptostreptococcus, Lachnospiraceae, and Parvimonas. In some embodiments, said probes are nucleic acid molecules having sequence complementarity with nucleic acid sequences of said plurality of populations of microbes. In some embodiments, said nucleic acid molecules are primers or enrichment sequences. In some embodiments, said assaying comprises use of array hybridization, polymerase chain reaction (PCR), or nucleic acid sequencing.

In some embodiments, said processing comprises assaying said biological sample using probes that are selective for said plurality of populations of microbes among other populations of microbes in said biological sample. In some embodiments, said probes are nucleic acid molecules having sequence complementarity with nucleic acid sequences of said plurality of populations of microbes. In some embodiments, said nucleic acid molecules are primers or enrichment sequences. In some embodiments, said assaying comprises use of array hybridization, polymerase chain reaction (PCR), or nucleic acid sequencing.

In another aspect, disclosed herein is a computer system for identifying or monitoring a progression or regression of a malignant colon condition in a subject, comprising: a database that is configured to store data indicative of a distribution of a plurality of populations of microbes of different types in a biological sample of said subject, wherein a presence, absence, or relative amount of individual populations of microbes of said plurality of populations of microbes is indicative of a malignant colon condition; and one or more computer processors operatively coupled to said database, wherein said one or more computer processors are individually collectively programmed to: (i) use a trained algorithm to process said data indicative of said distribution of said plurality of populations of microbes to determine a presence, absence, or relative amount of said individual populations of microbes of said plurality of populations of microbes in said biological sample, which trained algorithm is configured to identify said malignant colon condition with an accuracy of at least 90% for at least 100 independent samples; (ii) based on said presence, absence, or relative amount of said individual populations of microbes of said plurality of populations of microbes determined in (b), identify said subject as having said malignant colon condition with an accuracy of at least about 90%; and (iii) electronically output a report that identifies or provides an indication of said progression or regression of said malignant colon condition in said subject.

In some embodiments, the computer system further comprises an electronic display operatively coupled to said one or more computer processors, wherein said electronic display comprises a graphical user interface that is configured to display said report.

In another aspect, disclosed herein is a non-transitory computer readable medium comprising machine-executable code that, upon execution by one or more computer processors, implements a method for identifying or monitoring a progression or regression of a malignant colon condition in a subject, said method comprising: (a) obtaining data indicative of a distribution of a plurality of populations of microbes of different types in a biological sample of said subject, wherein a presence, absence, or relative amount of individual populations of microbes of said plurality of populations of microbes is indicative of a malignant colon condition; (b) using a trained algorithm to process said data indicative of said distribution of said plurality of populations of microbes to determine a presence, absence, or relative amount of said individual populations of microbes of said plurality of populations of microbes in said biological sample, which trained algorithm is configured to identify said malignant colon condition with an accuracy of at least 90% for at least 100 independent samples; (c) based on said presence, absence, or relative amount of said individual populations of microbes of said plurality of populations of microbes determined in (b), identifying said subject as having said malignant colon condition with an accuracy of at least about 90%; and (d) electronically outputting a report that identifies or provides an indication of said progression or regression of said malignant colon condition in said subject.

In another aspect, disclosed herein is a kit for identifying or monitoring a progression or regression of a malignant colon condition in a subject, comprising: probes for identifying a presence, absence, or relative amount of individual populations of microbes of a plurality of populations of microbes of different types in a biological sample of said subject, wherein a presence, absence, or relative amount of said individual populations of microbes of said plurality of populations of microbes in said biological is indicative of a malignant colon condition, wherein said probes are selective for said plurality of populations of microbes among other populations of microbes in said biological sample; and instructions for using said probes to process said biological sample to generate data indicative of a distribution of said plurality of populations of microbes of different types in said biological sample.

In some embodiments, said probes are selective for said plurality of populations of microbes among other populations of microbes in said biological sample. In some embodiments, said plurality of populations of microbes comprise at least 5 different populations of microbes. In some embodiments, said plurality of populations of microbes comprise at least 10 different populations of microbes. In some embodiments, said at least 5 different populations microbes are different species of microbes. In some embodiments, said at least 5 different species of microbes comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. In some embodiments, said plurality of populations of microbes comprise one or more members selected from the group consisting of Porphyromonas, Prevotella, Peptostreptococcus, Lachnospiraceae, and Parvimonas.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a Receiver Operator Characteristic (ROC) curve of a Random Forest classifier configured to identify colorectal cancer based on analysis of microbe populations in stool samples, in accordance with some embodiments.

FIG. 2A illustrates an example of mean abundance values of influential microbiome species (“top species”) that appear in at least 180 out of 200 runs of a Random Forest classifier configured to identify colorectal cancer based on analysis of microbe populations in stool samples, in accordance with some embodiments.

FIG. 2B illustrates an example of median abundance values of influential microbiome species (“top species”) that appear in at least 180 out of 200 runs of a Random Forest classifier configured to identify colorectal cancer based on analysis of microbe populations in stool samples, in accordance with some embodiments.

FIG. 3 illustrates a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a cell” includes a plurality of cells, including mixtures thereof.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets. Other examples of subjects include food, plant, soil, and water.

Biological samples (e.g., stool samples) obtained from subjects may be analyzed to measure microbiome distributions, e.g., a plurality of populations of microbes of different types in the biological sample. Such subjects may include subjects with malignant colon conditions (e.g., colorectal cancer) and subjects without these malignant colon conditions. Methods, systems, and kits are provided for detecting malignant colon conditions by processing biological samples indicative of a distribution of a plurality of populations of microbes of different types. A malignant colon condition may comprise colorectal cancer, colon cancer, or rectal cancer.

For some species of microbes, population measurements in cancerous samples (e.g., biological samples obtained from a subject with cancer) may be greater than in normal samples. For other species of microbes, population measurements in cancerous samples (e.g., biological samples obtained from a subject with cancer) may be less than in normal samples.

These species of microbes may be candidates for biomarkers for identifying colorectal cancer due to their differential presence in cancerous versus normal biological samples (“samples”). In particular, since collecting microbiome samples via feces is non-intrusive and next-generation sequencing is relatively inexpensive, microbiome distribution may be used as an early detection of colorectal cancer as an alternative to, or in conjunction with, traditional clinical tests such as colonoscopy or fecal immunochemical test (FIT). Microbiome distribution may be used to monitor a patient (e.g., subject who has colorectal cancer or who is being treated for colorectal cancer). In such cases, the microbiome distribution of the patient may change during the course of treatment. For example, the microbiome distribution of a patient whose colorectal cancer is regressing due to an effective treatment (e.g., chemotherapy or surgical resection) may shift toward the microbiome distribution of a healthy subject. Conversely, for example, the microbiome distribution of a patient whose colorectal cancer is progressing due to an ineffective treatment (e.g., when the tumor becomes resistant) may shift toward the microbiome distribution of a subject with more advanced stage colorectal cancer.

In an aspect, disclosed herein is a method for identifying or monitoring a progression or regression of a malignant colon condition in a subject. The method may comprise processing a biological sample obtained from the subject to generate data indicative of a distribution of a plurality of populations of microbes of different types in the biological sample. A presence, absence, or relative amount of individual populations of microbes of the plurality of populations of microbes may be indicative of a malignant colon condition. Next, a trained algorithm may be used to process the data indicative of the distribution of the plurality of populations of microbes to determine a presence, absence, or relative amount of the individual populations of microbes of the plurality of populations of microbes in the biological sample. The trained algorithm may be configured to identify the malignant colon condition with an accuracy of at least about 50%, 60%, 70%, 80%, 90%, 95%, or greater for at least about 10, 20, 30, 40, 50, 60, 70, 80, 90, 100, 150, 200, 250, or 300 independent samples. Next, based on the presence, absence, or relative amount of the individual populations of microbes of the plurality of populations of microbes, the subject may be identified as having the malignant colon condition with an accuracy of at least about 50%, 60%, 70%, 80%, 90%, 95%, or greater. A report may then be electronically outputted that identifies or provides an indication of the progression or regression of the malignant colon condition in the subject.

### Processing Biological Samples

The biological samples may comprise stool (feces) samples from a human subject. The stool samples may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, or potassium dichromate). The biological samples may comprise another source of gut microbiome from a human subject, such as re-suspended feces in fecal immunochemical test (FIT) cartridges.

The biological sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease or an age related disease. The infectious disease may be caused by bacteria, viruses, fungi and/or parasites. The cancer may be a colorectal cancer, a colon cancer, or a rectal cancer. The sample may be taken before and/or after treatment of a subject with a disease or disorder. Samples may be taken before and/or after a treatment. Samples may be taken during a treatment or a treatment regime. Multiple samples may be taken from a subject to monitor the effects of the treatment over time. The sample may be taken from a subject known or suspected of having a malignant colon condition for which a definitive positive or negative diagnosis is not available via clinical tests such as colonoscopy, biopsy, or fecal immunochemical test (FIT).

The sample may be taken from a subject suspected of having a disease or a disorder. The sample may be taken from a subject experiencing unexplained symptoms, such as fatigue, nausea, weight loss, aches and pains, weakness, or memory loss. The sample may be taken from a subject having explained symptoms. The sample may be taken from a subject at risk of developing a disease or disorder due to factors such as familial history, age, environmental exposure, lifestyle risk factors, or presence of other known risk factors.

After obtaining a biological sample from the subject, the biological sample obtained from the subject may be processed to generate data indicative of a distribution of a plurality of populations of microbes of different types in the biological sample. A presence, absence, or relative amount of individual populations of microbes of the plurality of populations of microbes may be indicative of a malignant colon condition. Processing the biological sample obtained from the subject may comprise (i) subjecting the biological sample to conditions that are sufficient to isolate the plurality of populations of microbes, and (ii) identifying the presence, absence, or relative amount of the individual populations of microbes of the plurality of populations of microbes.

The plurality of populations of microbes may be isolated by extracting nucleic acid molecules from the biological sample, and subjecting the nucleic acid molecules to sequencing to identify the presence, absence, or relative amount of the individual populations of microbes of the plurality of populations of microbes. The nucleic acid molecules may comprise deoxyribonucleic acid (DNA) or ribonucleic acid (RNA). The nucleic acid molecules may comprise DNA or RNA molecules of one or more microbial populations. The nucleic acid molecules (e.g., DNA or RNA) may be extracted from the biological sample by a variety of methods, such as a FastDNA Kit protocol from MP Biomedicals, a QIAamp DNA stool mini kit from Qiagen, or a stool DNA isolation kit protocol from Norgen Biotek. The extraction method may extract all DNA molecules from a sample. Alternatively, the extract method may selectively extract a portion of DNA molecules from a sample, e.g., by targeting certain genes such as 16S ribosomal RNA (rRNA) of one or more microbial species in the DNA molecules. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, RNA-Seq (Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci corresponding to one or more 16S ribosomal RNA (rRNA) genes.

The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA molecule may be traced back to the sample (and the subject) from which the DNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the data indicative of a distribution of a plurality of populations of microbes of different types in the biological sample. For example, the sequence reads may be aligned to one or more reference genomes (e.g., a genome of one or more bacterial species). The aligned sequence reads may be quantified at one or more genomic loci to generate the data indicative of a distribution of a plurality of populations of microbes of different types in the biological sample. For example, quantification of sequences corresponding to a plurality of conserved and/or non-conserved genomic loci may generate data indicative of a distribution of a plurality of populations of microbes of different types in the biological sample. Quantification of sequences may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genuses and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

The malignant colon condition may be identified or a progression or regression of the malignant colon condition may be monitored in the subject by using probes configured to selectively enrich nucleic acid (e.g., DNA or RNA) molecules corresponding to the individual populations of microbes. The probes may be nucleic acid primers. The probes may have sequence complementarity with nucleic acid sequences from one or more of the individual populations of microbes.

The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. The plurality of populations of microbes may comprise one or more members selected from the group consisting of Porphyromonas, Prevotella, Peptostreptococcus, Lachnospiraceae, and Parvimonas.

The biological sample may be processed to identify a distribution of a plurality of populations of microbes in the biological sample without any nucleic acid extraction. For example, the processing may comprise assaying the biological sample using probes that are selected for the plurality of populations of microbes. The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. The plurality of populations of microbes comprise one or more members selected from the group consisting of Porphyromonas, Prevotella, Peptostreptococcus, Lachnospiraceae, and Parvimonas.

The probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The assaying of the biological sample using probes that are selected for the plurality of populations of microbes may comprise use of array hybridization, polymerase chain reaction (PCR), or nucleic acid sequencing (e.g., DNA sequencing or RNA sequencing).

The processing may comprise assaying the biological sample using probes that are selective for the plurality of populations of microbes among other populations of microbes in the biological sample. These probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The assaying may comprise use of array hybridization, polymerase chain reaction (PCR), or nucleic acid sequencing (e.g., DNA sequencing or RNA sequencing).

The assay readouts may be quantified at one or more genomic loci to generate the data indicative of a distribution of a plurality of populations of microbes of different types in the biological sample. For example, quantification of array hybridization or polymerase chain reaction (PCR) corresponding to a plurality of conserved and/or non-conserved genomic loci may generate data indicative of a distribution of a plurality of populations of microbes of different types in the biological sample. Assay readouts may comprise quantitative PCR (qPCR) values, digital PCR (dPCR) values, digital droplet PCR (ddPCR) values, fluorescence values, etc. Quantification of array hybridization or polymerase chain reaction (PCR) may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genuses and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

### Kits

Provided herein are kits for identifying or monitoring a progression or regression of a malignant colon condition in a subject. A kit may comprise probes for identifying a presence, absence, or relative amount of individual populations of microbes of a plurality of populations of microbes of different types in a biological sample of the subject. A presence, absence, or relative amount of the individual populations of microbes of the plurality of populations of microbes in the biological may be indicative of a malignant colon condition. The probes may be selective for the plurality of populations of microbes among other populations of microbes in the biological sample. A kit may comprise instructions for using the probes to process the biological sample to generate data indicative of a distribution of the plurality of populations of microbes of different types in the biological sample.

The probes in the kit may be selective for the plurality of populations of microbes among other populations of microbes in the biological sample. The probes in the kit may be configured to selectively enrich nucleic acid (e.g., DNA or RNA) molecules corresponding to the individual populations of microbes. The probes in the kit may be nucleic acid primers. The probes in the kit may have sequence complementarity with nucleic acid sequences from one or more of the individual populations of microbes. The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus. The plurality of populations of microbes may comprise one or more members selected from the group consisting of Porphyromonas, Prevotella, Peptostreptococcus, Lachnospiraceae, and Parvimonas.

The instructions in the kit may comprise instructions to assay the biological sample using the probes that are selective for the plurality of populations of microbes among other populations of microbes in the biological sample. These probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The instructions to assay the biological sample may comprise introductions to perform array hybridization, polymerase chain reaction (PCR), or nucleic acid sequencing (e.g., DNA sequencing or RNA sequencing) to process the biological sample to generate data indicative of a distribution of a plurality of populations of microbes of different types in the biological sample. A presence, absence, or relative amount of individual populations of microbes of the plurality of populations of microbes may be indicative of a malignant colon condition.

The instructions in the kit may comprise instructions to measure and interpret assay readouts, which may be quantified at one or more genomic loci to generate the data indicative of a distribution of a plurality of populations of microbes of different types in the biological sample. For example, quantification of array hybridization or polymerase chain reaction (PCR) corresponding to a plurality of conserved and/or non-conserved genomic loci may generate data indicative of a distribution of a plurality of populations of microbes of different types in the biological sample. Assay readouts may comprise quantitative PCR (qPCR) values, digital PCR (dPCR) values, digital droplet PCR (ddPCR) values, fluorescence values, etc. Quantification of array hybridization or polymerase chain reaction (PCR) may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genuses and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

### Trained Algorithms

After processing a biological sample from the subject, a trained algorithm may be used to process the data indicative of the distribution of the plurality of populations of microbes (e.g., microbiome data) to determine a presence, absence, or relative amount of the individual populations of microbes of the plurality of populations of microbes in the biological sample. The trained algorithm may be configured to identify the malignant colon condition with an accuracy of at least 90% for at least 100 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise data indicative of the distribution of the plurality of populations of microbes (e.g., microbiome data). For example, an input variable may comprise data indicative of a distribution of a population of microbes (e.g., a bacterial genus or bacterial species).

The trained algorithm may comprise a classifier, such that each of the one or more output values comprises one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {cancerous, non-cancerous}) indicating a classification of the biological sample by the classifier. The trained algorithm may be another type of classifier, such that each of the one or more output values comprises one of more than two values (e.g., {0, 1, 2}, {positive, negative, or indeterminate}, or {cancerous, non-cancerous, or indeterminate}) indicating a classification of the biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, cancerous, non-cancerous, or indeterminate. Such descriptive labels may provide an identification of a treatment for the subject's disease or disorder state, and may comprise, for example, a therapeutic intervention, a duration of the therapeutic intervention, and/or a dosage of the therapeutic intervention. Such descriptive labels may provide an identification of secondary clinical tests that may be appropriate to perform on the subject, and may comprise, for example, a colonoscopy, a biopsy, a blood test, a fecal immunochemical test (FIT), a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease or disorder state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may comprise, for example, an un-normalized probability value of at least 0. Such continuous output values may indicate a prognosis of the disease or disorder state of the subject and may comprise, for example, an indication of an expected or average progression-free survival (PFS) or overall survival (OS) of the subject. Such continuous output values may indicate a prediction of the course of treatment to treat the disease or disorder state of the subject and may comprise, for example, an indication of an expected duration of efficacy of the course of treatment. Some numerical values may be mapped to descriptive labels, for example, by mapping 1 to “positive” and 0 to “negative”.

Some of the output values may be assigned based on one or more cutoff values. For example, a binary classification of samples may assign an output value of “positive” or 1 if the sample indicates that the subject has at least a 50% probability of being diseased. For example, a binary classification of samples may assign an output value of “negative” or 0 if the sample indicates that the subject has less than a 50% probability of being diseased. In this case, a single cutoff value of 50% is used to classify samples into one of the two possible binary output values. Examples of single cutoff values may include 1%, 2%, 5%, 10%, 15%, 20%, 25%, 30%, 35%, 40%, 45%, 50%, 55%, 60%, 65%, 70%, 75%, 80%, 85%, 90%, 95%, 98%, and 99%.

As another example, a classification of samples may assign an output value of “positive” or 1 if the sample indicates that the subject has a probability of being diseased of at least 50%, at least 55%, at least 60%, at least 65%, at least 70%, at least 75%, at least 80%, at least 85%, at least 90%, at least 95%, at least 98%, or at least 99%. The classification of samples may assign an output value of “positive” or 1 if the sample indicates that the subject has a probability of being diseased of more than 50%, more than 55%, more than 60%, more than 65%, more than 70%, more than 75%, more than 80%, more than 85%, more than 90%, more than 95%, more than 98%, or more than 99%. The classification of samples may assign an output value of “negative” or 0 if the sample indicates that the subject has a probability of being diseased of less than 50%, less than 45%, less than 40%, less than 35%, less than 30%, less than 25%, less than 20%, less than 10%, less than 5%, less than 2%, or less than 1%. The classification of samples may assign an output value of “negative” or 0 if the sample indicates that the subject has a probability of being diseased of no more than 50%, no more than 45%, no more than 40%, no more than 35%, no more than 30%, no more than 25%, no more than 20%, no more than 10%, no more than 5%, no more than 2%, or no more than 1%. The classification of samples may assign an output value of “indeterminate” or 2 if the sample has not been classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%} Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated data obtained by processing the biological sample (as described elsewhere herein), and one or more known output values corresponding to the biological sample (e.g., a clinical diagnosis, prognosis, treatment efficacy, or absence of a disease or disorder such as a malignant colon condition of the subject). Independent training samples may comprise biological samples and associated data and outputs obtained from a plurality of different subjects. Independent training samples may comprise biological samples and associated data and outputs obtained at a plurality of different time points from the same subject (e.g., before, after, and/or during a course of treatment to treat a disease or disorder of the subject). Independent training samples may be associated with presence of the malignant colon condition (e.g., training samples comprising biological samples and associated data and outputs obtained from a plurality of subjects known to have the malignant colon condition). Independent training samples may be associated with absence of the malignant colon condition (e.g., training samples comprising biological samples and associated data and outputs obtained from a plurality of subjects who are known to not have a previous diagnosis of the malignant colon condition, or otherwise who are asymptomatic for the malignant colon condition).

The trained algorithm may be trained with at least 50, at least 100, at least 150, at least 200, at least 250, at least 300, at least 350, at least 400, at least 450, or at least 500 independent training samples. The independent training samples may comprise samples associated with presence of the malignant colon condition and/or samples associated with absence of the malignant colon condition. The trained algorithm is trained with no more than 500, no more than 450, no more than 400, no more than 350, no more than 300, no more than 250, no more than 200, no more than 150, no more than 100, or no more than 50 independent training samples associated with presence of the malignant colon condition. In some embodiments, the biological sample is independent of samples used to train the trained algorithm.

The trained algorithm may be trained with a first number of independent training samples associated with presence of the malignant colon condition and a second number of independent training samples associated with absence of the malignant colon condition. The first number of independent training samples associated with presence of the malignant colon condition may be no more than the second number of independent training samples associated with absence of the malignant colon condition. The first number of independent training samples associated with presence of the malignant colon condition may be equal to the second number of independent training samples associated with absence of the malignant colon condition. The first number of independent training samples associated with presence of the malignant colon condition may be greater than the second number of independent training samples associated with absence of the malignant colon condition.

The trained algorithm may be configured to identify the malignant colon condition with an accuracy of at least 90%, at least 91%, at least 92%, at least 93%, at least 94%, at least 95%, at least 96%, at least 97%, at least 98%, or at least 99% for at least 100 independent samples. The trained algorithm may be configured to identify the malignant colon condition with an accuracy of at least 90%, at least 91%, at least 92%, at least 93%, at least 94%, at least 95%, at least 96%, at least 97%, at least 98%, or at least 99% for at least 150 independent samples. The trained algorithm may be configured to identify the malignant colon condition with an accuracy of at least 90%, at least 91%, at least 92%, at least 93%, at least 94%, at least 95%, at least 96%, at least 97%, at least 98%, or at least 99% for at least 200 independent samples. The trained algorithm may be configured to identify the malignant colon condition with an accuracy of at least 90%, at least 91%, at least 92%, at least 93%, at least 94%, at least 95%, at least 96%, at least 97%, at least 98%, or at least 99% for at least 250 independent samples. The trained algorithm may be configured to identify the malignant colon condition with an accuracy of at least 90%, at least 91%, at least 92%, at least 93%, at least 94%, at least 95%, at least 96%, at least 97%, at least 98%, or at least 99% for at least 300 independent samples. The accuracy of identifying the malignant colon condition by the trained algorithm may be calculated as the percentage of independent test samples (e.g., subjects known to have the malignant colon condition or apparently healthy subjects with negative clinical test results for the malignant colon condition) that are correctly identified or classified as having or not having the malignant colon condition.

The trained algorithm may be configured to identify the malignant colon condition with a positive predictive value (PPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The PPV of identifying the malignant colon condition by the trained algorithm may be calculated as the percentage of biological samples identified or classified as having the malignant colon condition that correspond to subjects that truly have the malignant colon condition. A PPV may also be referred to as a precision.

The trained algorithm may be configured to identify the malignant colon condition with a negative predictive value (NPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The NPV of identifying the malignant colon condition by the trained algorithm may be calculated as the percentage of biological samples identified or classified as not having the malignant colon condition that correspond to subjects that truly do not have the malignant colon condition.

The trained algorithm may be configured to identify the malignant colon condition with a clinical sensitivity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical sensitivity of identifying the malignant colon condition by the trained algorithm may be calculated as the percentage of independent test samples associated with presence of the malignant colon condition (e.g., subjects known to have the malignant colon condition) that are correctly identified or classified as having the malignant colon condition. A clinical sensitivity may also be referred to as a recall.

The trained algorithm may be configured to identify the malignant colon condition with a clinical specificity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical specificity of identifying the malignant colon condition by the trained algorithm may be calculated as the percentage of independent test samples associated with absence of the malignant colon condition (e.g., apparently healthy subjects with negative clinical test results for the malignant colon condition) that are correctly identified or classified as not having the malignant colon condition.

The trained algorithm may be configured to identify the malignant colon condition with an F-score of at least about 0.05, at least about 0.10, at least about 0.15, at least about 0.20, at least about 0.25, at least about 0.30, at least about 0.35, at least about 0.40, at least about 0.50, at least about 0.65, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. The F-score of identifying the malignant colon condition by the trained algorithm may be calculated as the harmonic mean of the precision and the recall of the identification.

The trained algorithm may be configured to identify the malignant colon condition with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. The AUC may be calculated as an integral of the Receiver Operator Characteristic (ROC) curve (e.g., the area under the ROC curve) associated with the trained algorithm in classifying biological samples as having or not having the malignant colon condition.

The trained algorithm may be adjusted or tuned to improve the performance, accuracy, PPV, NPV, clinical sensitivity, clinical specificity, or AUC of identifying the malignant colon condition. The trained algorithm may be adjusted or tuned by adjusting parameters of the trained algorithm (e.g., a set of cutoff values used to classify a sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high quality classifications. For example, if each input variable comprises data indicative of a distribution of a population of microbes (e.g., a bacterial genus or bacterial species), then a subset of the plurality of such input variables may be identified, indicating the populations of microbes that are most influential or most important to be included for making high quality classifications (e.g., an identification of a malignant colon condition). For example, for a Random Forest trained algorithm, metrics such as Mean Decrease Accuracy (MDA) and Mean Decrease Gini (MDG) may be used to evaluate the influence or importance of each input variable toward the classification performance. These metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, or AUC). For example, if training the training algorithm with a plurality comprising several dozen or hundreds of input variables in the trained algorithm results in an accuracy of classification of more than 99%, then training the training algorithm instead with only a selected subset of at most 5, at most 10, at most 15, or at most 20 such most influential or most important input variables among the plurality results in decreased but still acceptable accuracy of classification (e.g., at least 90% or at least 95%). The subset may be selected by rank-ordering the entire plurality of input variables by MDA and/or MDG and selecting a predetermined number (e.g., 5, 10, 15, or 20) of input variables with the highest values of MDA and/or MDG.

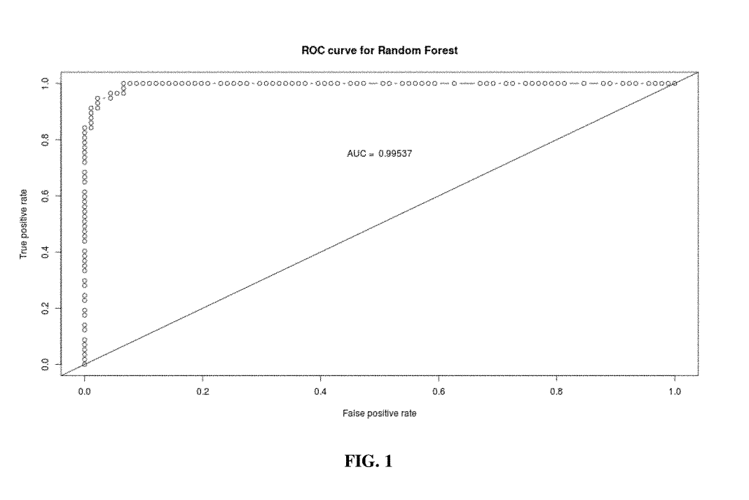

FIG. 1 illustrates an example of a Receiver Operator Characteristic (ROC) curve of a Random Forest (RF) classifier configured to identify colon cancer based on analysis of microbe populations in stool samples, in accordance with some embodiments. A Random Forest algorithm was used to analyze the relative abundance at the genus level of a data set of 295 biological samples obtained through Illumina HiSeq Paired-End sequencing based on 16S rRNA genes. The 295 microbiome samples comprised 187 samples that were obtained from normal subjects (e.g., diseased samples) and 108 samples that were obtained from patients with colon cancer (e.g., non-diseased samples). Analysis of sequencing data revealed that each of the biological samples comprised at least 614 different microbiome species.

The trained algorithm comprised a Random Forest classifier for predicting whether a sample is normal or cancerous, which was trained by performing a plurality of successive runs. For each of the plurality of successive runs, a training partition was performed, in which about half of the 295 biological samples were randomly selected as the training set (e.g., a set of independent training samples) for the Random Forest algorithm, and the other half (e.g., which was not previously selected for the training set) was designated as the testing set (e.g., a set of independent test samples). In total, 100 successive runs were performed to train the Random Forest classifier.

The average performance metrics of this Random Forest classifier were:

Mean accuracy ≥95%
Mean precision ˜0.95
Mean recall ˜0.96

### Mean F-Score ˜0.95

### Mean Area Under ROC Curve (AUC) ˜0.996

As further verification of the effectiveness of the Random Forest classifier, a blind-test data set comprising 18 cancerous samples and 18 normal samples were inputted into this trained Random Forest classifier (which was previously trained from the data set of 295 samples), and a prediction accuracy of >90% was observed. In particular, after careful tuning of the probability cutoff value based on the F-Score curve (e.g., by adjusting the probability cutoff value to increase the F-Score value as close to 1 as possible), 100% accuracy was achieved for this blind-test data.

Furthermore, after the Random Forest algorithm was trained, a consistent set of about 10 to 15 bacterial species was identified as being particularly influential for the Random Forest classifier performance, based on metrics such as Mean Decrease Accuracy (MDA) and Mean Decrease Gini (MDG). This represents a significant reduction in the number of input variables (e.g., predictor variables) from the original plurality of 614 bacterial species (e.g., populations of microbes). For example, after 200 runs were performed to train a Random Forest classifier, the following most influential bacterial species appeared in at least 180 of the runs (e.g., at least 90% of the total number of runs), as indicated in Table 1.

TABLE 1

| | Number of runs in which |
|---|---|
| Name of species | species appeared |
| Porphyromonas | 200 |
| Prevotella | 200 |
| Peptostreptococcus | 199 |
| Lachnospiraceae_UCG.001 | 196 |
| Parvimonas | 196 |
| Prevotella_2 | 190 |

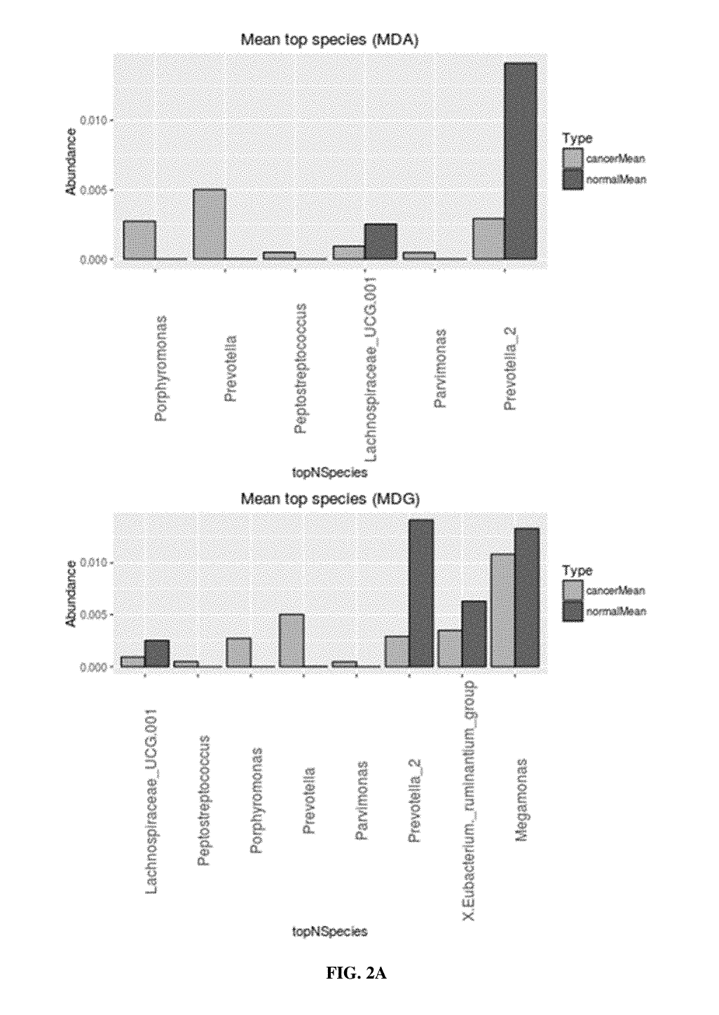

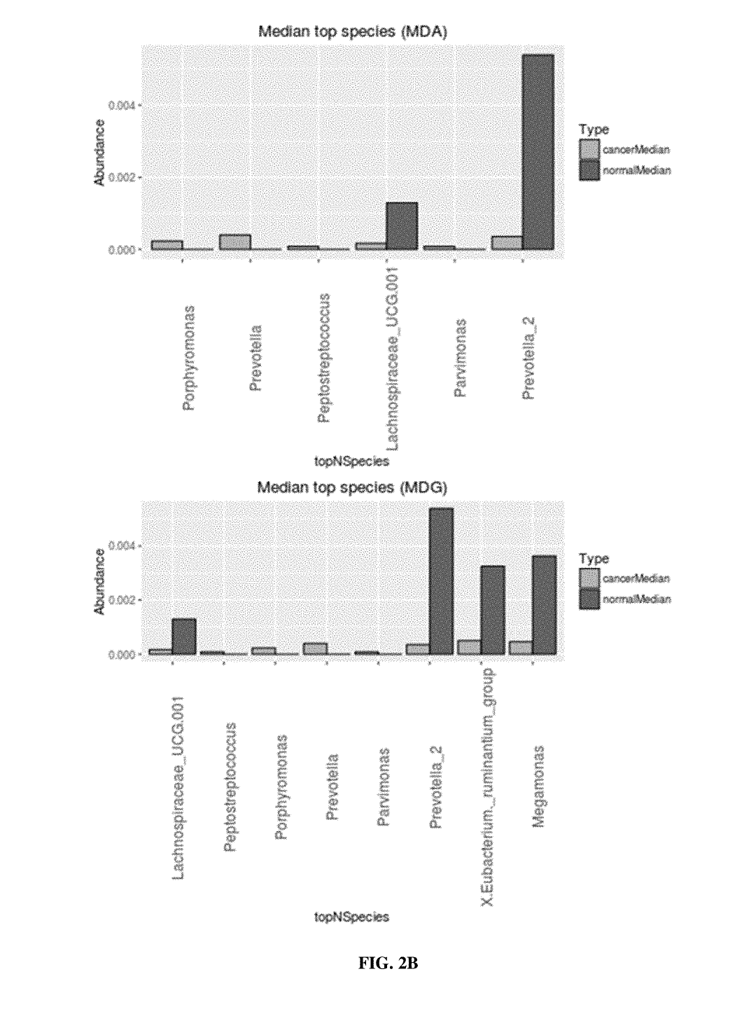

In addition, as seen in FIGS. 2A and 2B, the relative abundance of the above species display sharp contrasts between the normal samples and the cancerous samples, as measured by both mean and median values, respectively, across the samples for each species.

FIG. 2A illustrates an example of mean abundance values of influential microbiome species (“top species”) that appear in at least 180 out of 200 runs of a Random Forest classifier configured to identify colorectal cancer based on analysis of microbe populations in stool samples, in accordance with some embodiments. For each of the six top species by MDA (“Mean top species (MDA)”) shown here, a pair of bars illustrates the mean abundance for the top species, with the mean abundance for the cancerous sample shown on the left side of the pair in light gray shading (“cancerMean”) and the mean abundance for the normal sample shown on the right side of the pair in dark gray shading (“normalMean”) Similarly, for each of the eight top species by MDG (“Mean top species (MDG)”) shown here, a pair of bars illustrates the mean abundance for the top species, with the mean abundance for the cancerous sample shown on the left side of the pair in light gray shading (“cancerMean”) and the mean abundance for the normal sample shown on the right side of the pair in dark gray shading (“normalMean”).

FIG. 2B illustrates an example of median abundance values of influential microbiome species (“top species”) that appear in at least 180 out of 200 runs of a Random Forest classifier configured to identify colorectal cancer based on analysis of microbe populations in stool samples, in accordance with some embodiments. For each of the six top species by MDA (“Median top species (MDA)”) shown here, a pair of bars illustrates the median abundance for the top species, with the median abundance for the cancerous sample shown on the left side of the pair in light gray shading (“cancerMedian”) and the median abundance for the normal sample shown on the right side of the pair in dark gray shading (“normalMedian”). Similarly, for each of the eight top species by MDG (“Median top species (MDG)”) shown here, a pair of bars illustrates the median abundance for the top species, with the median abundance for the cancerous sample shown on the left side of the pair in light gray shading (“cancerMedian”) and the median abundance for the normal sample shown on the right side of the pair in dark gray shading (“normalMedian”).

For some of the above “top species” (e.g., populations of microbes), including Porphyromonas, Prevotella, Peptostreptococcus, and Parvimonas, the cancerous samples have greater relative abundance (for both mean and median values) than the normal samples. In contrast, for the other “top species”, including Lachnospiraceae_UCG.001, Prevotella_2, X.Eubacterium. ruminantium_group, and Megamonas, the normal samples have greater relative abundance (for both mean and median values) than the cancerous samples. Either or both of these types of top species may be potential candidates for biomarkers for identifying colorectal cancer.

To further investigate the relative importance of the top species identified by the MDA and MDG scores relative to the whole set of species, and their predictive ability if used by themselves as predictors (e.g., selected as a subset of the entire plurality of input variables for the Random Forest classifier), the mean MDA and MDG scores were measured for each species (out of a total of 614) over 5, 100, 200, and 500 runs. These MDA and MDG scores were then ranked in decreasing order. For each metric (MDA or MDG), a first subset (“TopHalf”) comprising a few species were identified, without which the Random Forest classifier's performance was reduced by half relative to its maximum. Furthermore, a second subset (“3Quarters_species”) comprising about one-sixth of the total species were identified, without which the Random Forest classifier's performance was reduced by 75% relative to its maximum. Note that the second subset is a superset of the first subset.

For analyzing the predictive power of each of these subsets of species, the first subset and the second subset of species were separately used as predictors by themselves to train a Random Forest classifier based on the existing samples. As before, a training partition was performed to randomly select about half of the samples as the training set, and the other half was designated as the test set. A Random Forest classifier trained using the first TopHalf subset was observed to have a typical accuracy of 93%, an AUC of 0.99, and a maximum F-score of 0.94. Similarly, this Random Forest classifier performance was slightly improved by training using the second 3Quarters_species subsets instead.

In particular, the first TopHalf subset of bacterial species identified using MDA (e.g., having the highest MDA values among the total set of input variables) comprised around 13-17 species across the various number of runs, while the corresponding subset identified using MDG comprised about 5-6 species across the various number of runs, as shown in Table 2.

TABLE 2

| Number of runs | TopHalf size (MDA) | TopHalf size (MDG) |
|---|---|---|
| 5 | 17 | 6 |
| 100 | 13 | 6 |
| 200 | 13 | 5 |
| 500 | 13 | 6 |

For instance, 6 species were identified as having the highest MDG values in the TopHalf subset (in decreasing order of importance), as shown in Table 3.

TABLE 3

Porphyromonas
Prevotella
Lachnospiraceae_UCG.001
Parvimonas
Peptostreptococcus
Prevotella_2

Note that the same 6 species were identified using the methods which produced the results summarized both Table 1 and Table 3.

As a negative test, a subset of species comprising negligible (e.g., nearly zero or negative) MDA or MDG scores was used to train a Random Forest classifier. This Random Forest classifier exhibited much worse performance: accuracy of 68%, AUC of 0.64, maximum F-score of 0.57. These performance metrics indicate that the Random Forest classifier trained using this subset of species has poor performance, not much better than the 50% accuracy of a completely random classifier (e.g., a classifier that produces outputs randomly).

### Identifying or Monitoring a Malignant Colon Condition

After using a trained algorithm to process the data indicative of the distribution of the plurality of populations of microbes, the malignant colon condition may be identified or a progression or regression of the malignant colon condition may be monitored in the subject by identifying the subject as having the malignant colon condition with an accuracy of at least about 90%. The identifying may be based on the presence, absence, or relative amount of the individual populations of microbes of the plurality of populations of microbes determined.

The malignant colon condition may be identified in the subject with an accuracy of at least 90%, at least 91%, at least 92%, at least 93%, at least 94%, at least 95%, at least 96%, at least 97%, at least 98%, or at least 99%. The accuracy of identifying the malignant colon condition by the trained algorithm may be calculated as the percentage of independent test samples (e.g., subjects known to have the malignant colon condition or apparently healthy subjects with negative clinical test results for the malignant colon condition) that are correctly identified or classified as having or not having the malignant colon condition.

The malignant colon condition may be identified in the subject with a positive predictive value (PPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The PPV of identifying the malignant colon condition by the trained algorithm may be calculated as the percentage of biological samples identified or classified as having the malignant colon condition that correspond to subjects that truly have the malignant colon condition. A PPV may also be referred to as a precision.

The malignant colon condition may be identified in the subject with a negative predictive value (NPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The NPV of identifying the malignant colon condition by the trained algorithm may be calculated as the percentage of biological samples identified or classified as not having the malignant colon condition that correspond to subjects that truly do not have the malignant colon condition.

The malignant colon condition may be identified in the subject with a clinical sensitivity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical sensitivity of identifying the malignant colon condition by the trained algorithm may be calculated as the percentage of independent test samples associated with presence of the malignant colon condition (e.g., subjects known to have the malignant colon condition) that are correctly identified or classified as having the malignant colon condition. A clinical sensitivity may also be referred to as a recall.

The malignant colon condition may be identified in the subject with a clinical specificity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical specificity of identifying the malignant colon condition by the trained algorithm may be calculated as the percentage of independent test samples associated with absence of the malignant colon condition (e.g., apparently healthy subjects with negative clinical test results for the malignant colon condition) that are correctly identified or classified as not having the malignant colon condition.

The malignant colon condition may be identified in the subject with an F-score of at least about 0.05, at least about 0.10, at least about 0.15, at least about 0.20, at least about 0.25, at least about 0.30, at least about 0.35, at least about 0.40, at least about 0.50, at least about 0.65, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. The F-score of identifying the malignant colon condition by the trained algorithm may be calculated as the harmonic mean of the precision and the recall of the identification.

After the colorectal cancer is identified in a subject, a stage of the colorectal cancer (e.g., stage I, stage II, stage III, or stage IV) may further be identified. The stage of the colorectal cancer may be determined based on the presence, absence, or relative amount of the individual populations of microbes of the plurality of populations of microbes determined.

Upon identifying the subject as having the malignant colon condition, the subject may be provided with a therapeutic intervention (e.g., prescribing an appropriate course of treatment to treat the malignant colon condition of the subject). The therapeutic intervention may comprise a surgical tumor resection, an effective dose of chemotherapy, an effective dose of radiotherapy, an effective dose of targeted therapy, an effective dose of immunotherapy. If the subject is currently being treated for the malignant colon condition with a course of treatment, the therapeutic intervention may comprise a subsequent different course of treatment (e.g., to increase treatment efficacy due to tumor resistance, tumor recurrence, non-response of the current course of treatment).

The therapeutic intervention may comprise recommending the subject for a secondary clinical test to confirm a diagnosis of the malignant colon condition. This secondary clinical test may comprise a colonoscopy, a biopsy, a blood test, a fecal immunochemical test (FIT), a computed tomography (CT) scan, a magnetic resonance imaging (MRI) scan, an ultrasound scan, a chest X-ray, a positron emission tomography (PET) scan, or a PET-CT scan.

The subject may be treated upon identifying the subject as having the malignant colon condition. Treating the subject may comprise administering an appropriate therapeutic intervention to treat the malignant colon condition of the subject. The therapeutic intervention may comprise a surgical tumor resection, an effective dose of chemotherapy, an effective dose of radiotherapy, an effective dose of targeted therapy, an effective dose of immunotherapy. If the subject is currently being treated for the malignant colon condition with a course of treatment, the administered therapeutic intervention may comprise a subsequent different course of treatment (e.g., to increase treatment efficacy due to tumor resistance, tumor recurrence, non-response of the current course of treatment).

Microbiome distributions in a biological sample may be used to monitor a patient (e.g., subject who has colorectal cancer or who is being treated for colorectal cancer). In such cases, the microbiome distribution of the patient may change during the course of treatment. For example, the microbiome distribution of a patient whose colorectal cancer is regressing due to an effective treatment (e.g., chemotherapy or surgical resection) may shift toward the microbiome distribution of a healthy subject. Conversely, for example, the microbiome distribution of a patient whose colorectal cancer is progressing due to an ineffective treatment (e.g., when the tumor becomes resistant) may shift toward the microbiome distribution of a subject with more advanced stage colorectal cancer.

The progression or regression of the malignant colon condition in the subject may be monitored by monitoring a course of treatment for treating the malignant colon condition in the subject. The monitoring may comprise assessing the malignant colon condition in the subject at two or more time points. The assessing may be based at least on the presence, absence, or relative amount of the individual populations of microbes of the plurality of populations of microbes determined at each of the two or more time points.

A difference in the presence, absence, or relative amount of the individual populations of microbes of the plurality of populations of microbes determined between the two or more time points may be indicative of one or more clinical indications, such as (i) a diagnosis of the malignant colon condition in the subject, (ii) a prognosis of the malignant colon condition in the subject, (iii) a progression of the malignant colon condition in the subject, (iv) a regression of the malignant colon condition in the subject, (v) an efficacy of the course of treatment for treating the malignant colon condition in the subject, and (vi) a resistance of the malignant colon condition toward the course of treatment for treating the malignant colon condition in the subject.

A difference in the presence, absence, or relative amount of the individual populations of microbes of the plurality of populations of microbes determined between the two or more time points may be indicative of a diagnosis of the malignant colon condition in the subject. For example, if the malignant colon condition was not detected in the subject at an earlier time point but was detected in the subject at a later time point, then the difference is indicative of a diagnosis of the malignant colon condition in the subject. A clinical action or decision may be made based on this indication of diagnosis of the malignant colon condition in the subject, e.g., prescribing a new therapeutic intervention for the subject.

A difference in the presence, absence, or relative amount of the individual populations of microbes of the plurality of populations of microbes determined between the two or more time points may be indicative of a prognosis of the malignant colon condition in the subject.

A difference in the presence, absence, or relative amount of the individual populations of microbes of the plurality of populations of microbes determined between the two or more time points may be indicative of a progression of the malignant colon condition in the subject. For example, if the malignant colon condition was detected in the subject both at an earlier time point and at a later time point, and if the difference is a negative difference (e.g., the presence, absence, or relative amount of the individual populations of microbes of the plurality of populations of microbes increased from the earlier time point to the later time point), then the difference may be indicative of a progression (e.g., increased tumor load, tumor burden, or tumor size) of the malignant colon condition in the subject. A clinical action or decision may be made based on this indication of the progression, e.g., prescribing a new therapeutic intervention or switching therapeutic interventions (e.g., ending a current treatment and prescribing a new treatment) for the subject.

A difference in the presence, absence, or relative amount of the individual populations of microbes of the plurality of populations of microbes determined between the two or more time points may be indicative of a regression of the malignant colon condition in the subject. For example, if the malignant colon condition was detected in the subject both at an earlier time point and at a later time point, and if the difference is a positive difference (e.g., the presence, absence, or relative amount of the individual populations of microbes of the plurality of populations of microbes decreased from the earlier time point to the later time point), then the difference may be indicative of a regression (e.g., decreased tumor load, tumor burden, or tumor size) of the malignant colon condition in the subject. A clinical action or decision may be made based on this indication of the regression, e.g., continuing or ending a current therapeutic intervention for the subject.

A difference in the presence, absence, or relative amount of the individual populations of microbes of the plurality of populations of microbes determined between the two or more time points may be indicative of an efficacy of the course of treatment for treating the malignant colon condition in the subject. For example, if the malignant colon condition was detected in the subject at an earlier time point but was not detected in the subject at a later time point, then the difference may be indicative of an efficacy of the course of treatment for treating the malignant colon condition in the subject. A clinical action or decision may be made based on this indication of the efficacy of the course of treatment for treating the malignant colon condition in the subject, e.g., continuing or ending a current therapeutic intervention for the subject.

A difference in the presence, absence, or relative amount of the individual populations of microbes of the plurality of populations of microbes determined between the two or more time points may be indicative of a resistance of the malignant colon condition toward the course of treatment for treating the malignant colon condition in the subject. For example, if the malignant colon condition was detected in the subject both at an earlier time point and at a later time point, and if the difference is a negative or zero difference (e.g., the presence, absence, or relative amount of the individual populations of microbes of the plurality of populations of microbes increased or remained at a constant level from the earlier time point to the later time point), and if an efficacious treatment was indicated at an earlier time point, then the difference may be indicative of a resistance (e.g., increased or constant tumor load, tumor burden, or tumor size) of the course of treatment for treating the malignant colon condition in the subject. A clinical action or decision may be made based on this indication of the resistance of the course of treatment for treating the malignant colon condition in the subject, e.g., ending a current therapeutic intervention and/or switching to (e.g., prescribing) a different new therapeutic intervention for the subject.

### Outputting a Report of the Malignant Colon Condition

After the malignant colon condition is identified or a progression or regression of the malignant colon condition is monitored in the subject, a report may be electronically outputted that identifies or provides an indication of the progression or regression of the malignant colon condition in the subject. The subject may not display a benign or malignant colon condition (e.g., is asymptomatic of the benign or malignant colon condition). The report may be presented on a graphical user interface (GUI) of an electronic device of a user. The user may be the subject, a caretaker, a physician, a nurse, or another health care worker.

The report may include one or more clinical indications such as (i) a diagnosis of the malignant colon condition in the subject, (ii) a prognosis of the malignant colon condition in the subject, (iii) a progression of the malignant colon condition in the subject, (iv) a regression of the malignant colon condition in the subject, (v) an efficacy of the course of treatment for treating the malignant colon condition in the subject, and (vi) a resistance of the malignant colon condition toward the course of treatment for treating the malignant colon condition in the subject. The report may include one or more clinical actions or decisions made based on these one or more clinical indications.

For example, a clinical indication of a diagnosis of the malignant colon condition in the subject may be accompanied with a clinical action of prescribing a new therapeutic intervention for the subject. As another example, a clinical indication of a progression of the malignant colon condition in the subject may be accompanied with a clinical action of prescribing a new therapeutic intervention or switching therapeutic interventions (e.g., ending a current treatment and prescribing a new treatment) for the subject. As another example, a clinical indication of a regression of the malignant colon condition in the subject may be accompanied with a clinical action of continuing or ending a current therapeutic intervention for the subject. As another example, a clinical indication of an efficacy of the course of treatment for treating the malignant colon condition in the subject may be accompanied with a clinical action of continuing or ending a current therapeutic intervention for the subject. As another example, a clinical indication of a resistance of the course of treatment for treating the malignant colon condition in the subject may be accompanied with a clinical action of ending a current therapeutic intervention and/or switching to (e.g., prescribing) a different new therapeutic intervention for the subject.

### Computer Control Systems

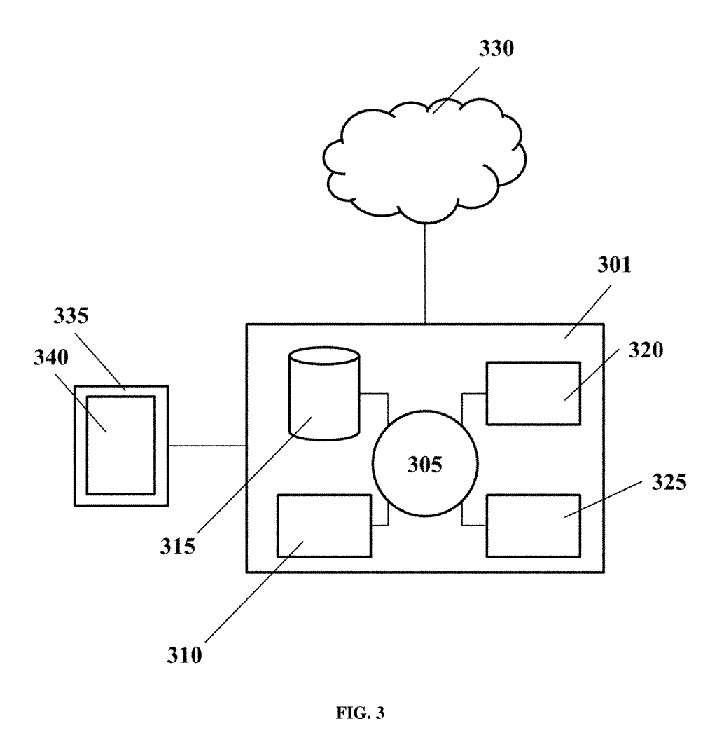

The present disclosure provides computer control systems that are programmed to implement methods of the disclosure. FIG. 3 shows a computer system 301 that is programmed or otherwise configured to, for example, (i) train and test a trained algorithm, (ii) use the trained algorithm to process data indicative of a distribution of a plurality of populations of microbes, (iii) determine a presence, absence, or relative amount of the individual populations of microbes of the plurality of populations of microbes in the biological sample, (iv) identify the subject as having the malignant colon condition, or (v) electronically output a report that identifies or provides an indication of the progression or regression of the malignant colon condition in the subject.

The computer system 301 can regulate various aspects of analysis, calculation, and generation of the present disclosure, such as, for example, (i) training and testing a trained algorithm, (ii) using the trained algorithm to process data indicative of a distribution of a plurality of populations of microbes, (iii) determining a presence, absence, or relative amount of the individual populations of microbes of the plurality of populations of microbes in the biological sample, (iv) identifying the subject as having the malignant colon condition, or (v) electronically outputting a report that identifies or provides an indication of the progression or regression of the malignant colon condition in the subject. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet.

The network 330 in some cases is a telecommunication and/or data network. The network 330 can include one or more computer servers, which can enable distributed computing, such as cloud computing. For example, one or more computer servers may enable cloud computing over the network 330 (“the cloud”) to perform various aspects of analysis, calculation, and generation of the present disclosure, such as, for example, (i) training and testing a trained algorithm, (ii) using the trained algorithm to process data indicative of a distribution of a plurality of populations of microbes, (iii) determining a presence, absence, or relative amount of the individual populations of microbes of the plurality of populations of microbes in the biological sample, (iv) identifying the subject as having the malignant colon condition, or (v) electronically outputting a report that identifies or provides an indication of the progression or regression of the malignant colon condition in the subject. Such cloud computing may be provided by cloud computing platforms such as, for example, Amazon Web Services (AWS), Microsoft Azure, Google Cloud Platform, and IBM cloud. The network 330, in some cases with the aid of the computer system 301, can implement a peer-to-peer network, which may enable devices coupled to the computer system 301 to behave as a client or a server.

The CPU 305 may comprise one or more computer processors and/or one or more graphics processing units (GPUs). The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, (i) a visual display indicative of training and testing of a trained algorithm, (ii) a visual display of data indicative of a distribution of a plurality of populations of microbes, (iii) a determined presence, absence, or relative amount of the individual populations of microbes of the plurality of populations of microbes in the biological sample, (iv) an identification of the subject as having the malignant colon condition, or (v) an electronic report that identifies or provides an indication of the progression or regression of the malignant colon condition in the subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, (i) train and test a trained algorithm, (ii) use the trained algorithm to process data indicative of a distribution of a plurality of populations of microbes, (iii) determine a presence, absence, or relative amount of the individual populations of microbes of the plurality of populations of microbes in the biological sample, (iv) identify the subject as having the malignant colon condition, or (v) electronically output a report that identifies or provides an indication of the progression or regression of the malignant colon condition in the subject.

Methods and systems of the present disclosure may be combined with or modified by other methods and systems, such as, for example, those described in PCT/CN2013/090425 and PCT/CN2015/095763, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-73. (canceled)

74. A method for identifying or monitoring a progression or regression of a malignant colon condition in a subject, comprising:
(a) processing a biological sample obtained from said subject to generate data indicative of a distribution of a plurality of populations of microbes of different types in said biological sample, wherein a presence, absence, or relative amount of individual populations of microbes of said plurality of populations of microbes is indicative of a malignant colon condition;
(b) using a trained algorithm to process said data indicative of said distribution of said plurality of populations of microbes to determine a presence, absence, or relative amount of said individual populations of microbes of said plurality of populations of microbes in said biological sample, which trained algorithm is configured to identify said malignant colon condition with an accuracy of at least 90% for at least 100 independent samples;
(c) based on said presence, absence, or relative amount of said individual populations of microbes of said plurality of populations of microbes determined in (b), identifying said subject as having said malignant colon condition with an accuracy of at least about 90%; and
(d) electronically outputting a report that identifies or provides an indication of said progression or regression of said malignant colon condition in said subject.

75. The method of claim 74, wherein said biological sample is independent of samples used to train said trained algorithm.

76. The method of claim 74, wherein said trained algorithm is configured to identify said malignant colon condition with a positive predictive value (PPV) of at least about 70%.

77. The method of claim 74, wherein said trained algorithm is configured to identify said malignant colon condition with a clinical sensitivity of at least about 90%.

78. The method of claim 74, wherein said trained algorithm is configured to identify said malignant colon condition with an Area Under Curve (AUC) of at least about 0.90.

79. The method of claim 74, wherein said biological sample is feces.

80. The method of claim 74, wherein said trained algorithm is trained with no more than 200 independent training samples associated with presence of said malignant colon condition.

81. The method of claim 74, wherein said trained algorithm is trained with a first number of independent training samples associated with presence of said malignant colon condition and a second number of independent training samples associated with absence of said malignant colon condition, wherein the first number is no more than the second number.

82. The method of claim 74, wherein (a) comprises (i) subjecting said biological sample to conditions that are sufficient to isolate said plurality of populations of microbes, and (ii) identifying said presence, absence, or relative amount of said individual populations of microbes of said plurality of populations of microbes.

83. The method of claim 82, further comprising extracting nucleic acid molecules from said biological sample, and subjecting said nucleic acid molecules to sequencing to identify said presence, absence, or relative amount of said individual populations of microbes of said plurality of populations of microbes.

84. The method of claim 83, wherein said sequencing comprises nucleic acid amplification.

85. The method of claim 83, further comprising using probes configured to selectively enrich nucleic acid molecules corresponding to said individual populations of microbes.

86. The method of claim 74, wherein said plurality of populations of microbes comprise at least 5 different populations of microbes.

87. The method of claim 86, wherein said at least 5 different populations microbes are different species of microbes.

88. The method of claim 87, wherein said at least 5 different species of microbes comprise one or more members selected from the group consisting of Prevotella intermedia, Porphyromonas asaccharolytica, Dialister pneumosintes, Porphyromonas endodontalis, Parasutterella secunda, Alloprevotella tannerae, Roseburia intestinalis, and Ruminococcus callidus.

89. The method of claim 86, wherein said plurality of populations of microbes comprise one or more members selected from the group consisting of Porphyromonas, Prevotella, Peptostreptococcus, Lachnospiraceae, and Parvimonas.

90. The method of claim 74, wherein said malignant colon condition is colorectal cancer.

91. The method of claim 74, wherein said trained algorithm comprises a supervised machine learning algorithm.

92. The method of claim 91, wherein said supervised machine learning algorithm comprises a Random Forest, a support vector machine (SVM), a neural network, or a deep learning algorithm.

93. A computer system for identifying or monitoring a progression or regression of a malignant colon condition in a subject, comprising:
a database that is configured to store data indicative of a distribution of a plurality of populations of microbes of different types in a biological sample of said subject, wherein a presence, absence, or relative amount of individual populations of microbes of said plurality of populations of microbes is indicative of a malignant colon condition; and
one or more computer processors operatively coupled to said database, wherein said one or more computer processors are individually collectively programmed to:
(i) use a trained algorithm to process said data indicative of said distribution of said plurality of populations of microbes to determine a presence, absence, or relative amount of said individual populations of microbes of said plurality of populations of microbes in said biological sample, which trained algorithm is configured to identify said malignant colon condition with an accuracy of at least 90% for at least 100 independent samples;
(ii) based on said presence, absence, or relative amount of said individual populations of microbes of said plurality of populations of microbes determined in (b), identify said subject as having said malignant colon condition with an accuracy of at least about 90%; and
(iii) electronically output a report that identifies or provides an indication of said progression or regression of said malignant colon condition in said subject.

94. A kit for identifying or monitoring a progression or regression of a malignant colon condition in a subject, comprising:
probes for identifying a presence, absence, or relative amount of individual populations of microbes of a plurality of populations of microbes of different types in a biological sample of said subject, wherein a presence, absence, or relative amount of said individual populations of microbes of said plurality of populations of microbes in said biological is indicative of a malignant colon condition, wherein said probes are selective for said plurality of populations of microbes among other populations of microbes in said biological sample; and
instructions for using said probes to process said biological sample to generate data indicative of a distribution of said plurality of populations of microbes of different types in said biological sample.
